(19) United States
(12) Patent Application Publication
(10) Pub. No.: US 2019/0314082 A1
(43) Pub. Date: Oct. 17, 2019

(54) Electrosurgical Instrument

(71) Applicants: Brandon J. Shuman, Redmond, WA (US); Peter A. Lambe, Redmond, WA (US); Christopher A. Saleira, Redmond, WA (US); Zoie R. Engman, Redmond, WA (US); Spiretion Inc. d/b/a Olympus Respiratory America, Redmond, WA (US)

(72) Inventors: Brandon J. Shuman, Kirkland, WA (US); Peter A. Lambe, Redmond, WA (US); Christopher A. Saleira, Bothell, WA (US); Zoie R. Engman, Kirkland, WA (US)

(21) Appl. No.: 16/346,756

(22) PCT Filed: Nov. 4, 2016
(86) PCT No.: PCT/US16/60633

§ 371 Date: May 1, 2019

(51) Int. Cl. A61B 18/14

(52) U.S. Cl. CPC A61B 18/1477; A61B 2018/00589

(57) ABSTRACT

An electrosurgical instrument includes a needle configured as a first electrode and a coil extending through the needle and configured as a second electrode. The coil movable relative to the needle, and as the needle and the coil are inserted into tissue and energized with an electrical energy source, the needle and the coil apply current to the tissue to coagulate the tissue. The coil includes a fluid transmission feature that allows a fluid to flow distally for cooling the coil.

### FIELD

The present invention relates to an electrosurgical instrument for treating tissue.

### BACKGROUND

The statements in this section merely provide background information related to the present disclosure and may not constitute prior art.

Certain electrosurgical instruments used for treating tissue generally include a guide catheter and an applicator inserted through the catheter. These electrosurgical instruments are usually inserted into a body lumen to place the distal end of the applicator at a desired location. The applicator generally includes one or more electrodes at the distal end. Such electrodes emit a radiofrequency (RF) signal to surrounding tissue to coagulate or ablate the tissue. Monopolar electrosurgical instruments only require one electrode that interacts with a neutral electrode, which is likewise connected to the body of a patient. A bipolar electrosurgical instrument typically includes an applicator with two electrodes (a distal electrode and a proximal electrode). A RF voltage with different potentials is applied to such bipolar instruments so that a current passes from one electrode to the other electrode through the tissue, thereby heating the tissue to coagulate or ablate the tissue.

During and after the treatment of tissue with the above-described electrosurgical instruments, there is a desire to cool the electrodes. Overheated electrodes produce undesirable effects.

### SUMMARY

The present invention provides an electrosurgical instrument for treating tissue, for example, ablating or coagulating tissue.

Accordingly, pursuant to one aspect of the present invention, an electrosurgical instrument includes a needle configured as a first electrode and a coil extending through the needle and configured as a second electrode. The coil is movable relative to the needle. As the needle and the coil are inserted into tissue and energized with an electrical energy source, the needle and the coil apply energy to the tissue to coagulate the tissue. The coil includes one or more cooling features that allow a cooling fluid to remain in contact, thus aiding in rapid cooling of the coil.

Accordingly, pursuant to another aspect of the present invention, a straight section of the coil includes a fluid delivery path identified by a first side of the straight section and a gap formed by a material.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 illustrates an overview of a system for treating tissue in accordance with the principles of the present invention;

FIG. 2 illustrates a close-up view of the distal end of an insertion tube of the system shown in FIG. 1;

FIG. 3-1 illustrates a side view of the distal end of the needle and coil formed in accordance with an embodiment of the present invention;

FIG. 3-2 illustrates a perspective view of a coil formed in accordance with an embodiment of the present invention;

FIG. 3-3 illustrates a partial view of a first side of the coil of FIG. 3-1 or 3-2;

FIG. 3-4 illustrates a partial side x-ray view of a second side of the coil of FIG. 3-1 or 3-2;

FIG. 3-5 illustrates a cross-sectional view of the coil of FIG. 3-1 or 3-2;

FIG. 4-1 illustrates a partial view of a first side of a coil formed in accordance with an embodiment of the present invention;

FIG. 4-2 illustrates a cross-sectional view of the coil of FIG. 4-1;

FIG. 5-1 illustrates a partial view of a first side of a coil formed in accordance with an embodiment of the present invention;

FIG. 5-2 illustrates a cross-sectional view of the coil of FIG. 5-1;

FIG. 6-1 illustrates a partial view of a first side of a coil formed in accordance with an embodiment of the present invention;

FIG. 6-2 illustrates a cross-sectional view of the coil of FIG. 6-1;

FIG. 7 illustrates a cross-sectional view of a coil formed in accordance with an embodiment of the present invention;

FIG. 8 illustrates a cross-sectional view of a coil formed in accordance with an embodiment of the present invention;

FIG. 9 illustrates a cross-sectional view of a coil formed in accordance with an embodiment of the present invention;

FIG. 10-1 illustrates a partial view of the straight section of a coil formed in accordance with an embodiment of the present invention;

FIG. 10-2 illustrates a cross-sectional view of a first embodiment associated with the coil shown in FIG. 10-1;

FIG. 10-3 illustrates a cross-sectional view of a second embodiment associated with the coil shown in FIG. 10-1; and

FIG. 10-4 illustrates a cross-sectional view of another embodiment associated with the coil shown in FIG. 10-1.

### DETAILED DESCRIPTION

The following description is merely exemplary in nature and is not intended to limit the present disclosure, application, or uses.

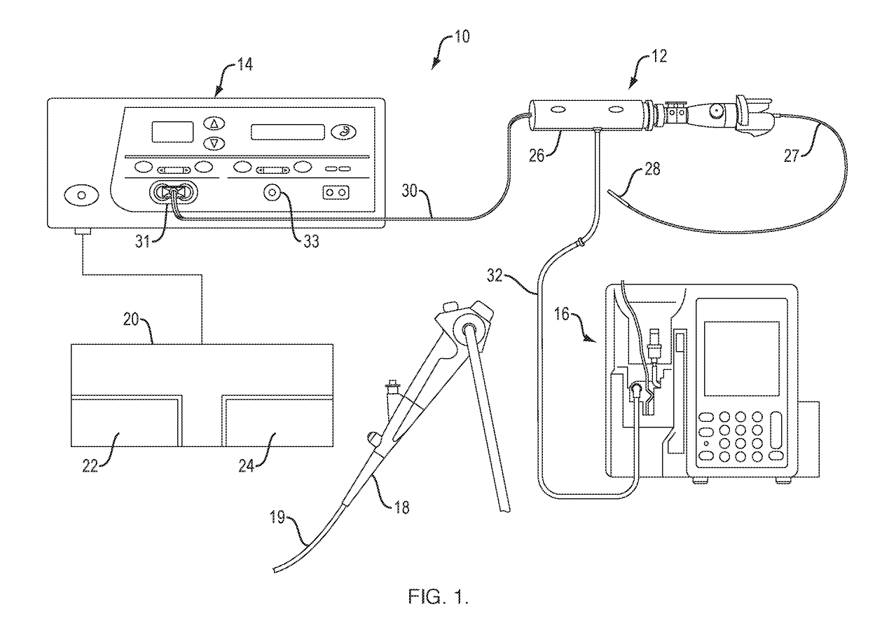

Referring now to FIG. 1, a system 10 for treating tissue in an anatomical region of a patient is illustrated. The system 10 is a monopolar/bipolar radio frequency (RF) system for treating tissue in a patient. Specifically, the system 10 is employed for coagulation and ablation of soft tissue during percutaneous or endoscopic procedures, including bronchoscopy and surgical procedures, such as, for example, partial or complete ablation of organ lesions.

In some arrangements, the system 10 includes an applicator 12, an electrosurgical RF generator 14, an infusion pump 16, and a bronchoscope 18. The applicator 12 electrically communicates with the generator 14 through a lead 30. The lead 30 is connected to a generator outlet 31 when the system 10 is operated in a bipolar mode. Alternatively, the system 10 can be operated in a monopolar mode when the lead 30 is connected to an outlet 33 with an adapter as necessary. The applicator 12 is further connected to the infusion pump 16 with a tube 32 that facilitates the flow of liquid, for example a saline solution, from the pump 16 to the applicator 12. The applicator 12 includes a handle 26 and a needle 28 and a coil (not shown) that extends from the handle 26 through a sheath 27.

The generator 14 can be operated with the use of a foot operated unit 20 electrically connected to the generator 14. The foot operated unit 20 includes a pedal 22 that instructs the generator 14 to apply a RF potential to electrodes (described below) to cut or ablate tissue and a pedal 24 that instructs the generator 14 to apply a different RF potential to the electrodes to coagulate tissue.

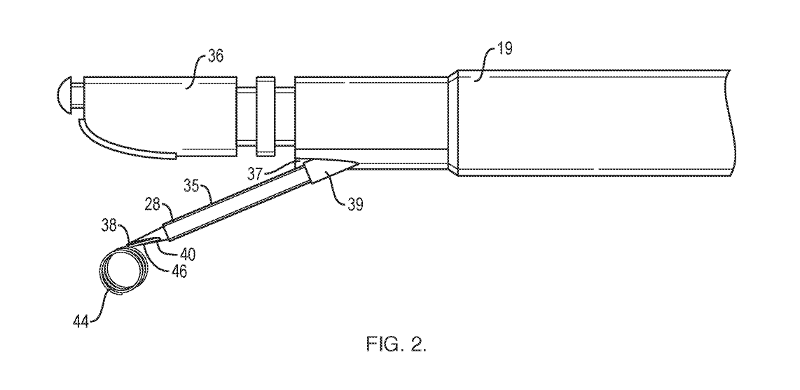

The bronchoscope 18 includes an insertion tube 19. As shown in FIG. 2, a distal end 36 of the insertion tube 19 includes an opening 37. In certain procedures, the needle 28 and the sheath 27 are inserted into the bronchoscope 18 through the insertion tube 19 so that a distal end of the needle 28 exits the distal end 36 through the opening 37.

A coil 44 from the applicator 12 extends through the needle 28 to exit an opening 40 at the distal end of the needle 28. The needle 28 may include an exterior layer of insulation 35 (e.g., a thermoplastic elastomer or a polymer heat shrink material, such as Pebax®) and the coil 44 may include an exterior layer of insulation 46 to electrically isolate the needle 28 from the coil 44. Accordingly, in this arrangement, the needle 28 operates as a proximal electrode and the coil 44 operates as a distal electrode when the system 10 is operated in a bipolar mode.

The needle 28 also includes a tip 38 for piercing tissue. Specifically, during the penetration of the needle 28 into tissue, only the needle 28 (that is, not the coil 44) may be energized in a monopolar mode (for example, with the patient grounded to a patient pad to complete the circuit) with the generator 14 set at a power level that would be lower than that used in a bipolar mode.

An example procedure for using the system 10 includes a physician advancing the insertion tube 19 of the bronchoscope 18 through a passageway, for example, an airway, until the distal end 36 is positioned near target tissue to be treated. The physician then inserts the needle 28 into the insertion tube 19 and advances the needle 28 until the needle 28 exits the opening 37 and penetrates into the target tissue with the tip 38. Next the physician advances the coil 44 through the needle 28 until it exits the opening 40. The physician continues to advance the coil 44 coiling as it advances. Once the coil 44 has been deployed, bipolar ablation can occur before or after the needle 28 is retracted proximally.

To energize the electrodes (the needle 28, the coil 44) for coagulating the target tissue, the physician sets the generator 14 to a desired power level and pushes the pedal 24 of the foot unit 20 to apply a RF potential to the electrodes. As such, RF electrical current passes between the needle 28 and the coil 44 through the target tissue. The level of RF electrical current is set by the physician to control the desired extent of the coagulation region in the target tissue. To ablate or cut tissue, the physician pushes the pedal 22 of the foot unit 20 to apply a different RF potential to the electrodes. Note that anytime during the procedure, the physician can activate the infusion pump 16 to supply saline solution to the applicator 12 so that the saline solution flows through the needle 28 to the location of interest in the target tissue. The saline solution is employed to cool the electrodes (the needle 28 and/or the coil 44) and to prevent dehydration of the target tissue. The cooling fluid is wicked (i.e., capillary action) via one or more grooves on a surface of the coil 44. The fluid cools the needle 28 and coil 44 as it wicks along at least the curved portion of the coil 44.

After treatment of the target tissue is completed, the physician turns off the generator 14, moves the needle 28 forward to the position relative to the coil 44 prior to coil deployment, if it is not already at this most distal position. Then, the coil 44 is retracted into the needle 28. Then, the needle 28 and the coil 44 are retracted into the insertion tube 19 within the bronchoscope 18. The bronchoscope 18 is withdrawn from the patient to complete the procedure.

The needle 28 is made from any suitable material, such as, for example, stainless steel that enables penetration into tissue. In various arrangements, all or a portion of the coil 44 is made from a shape memory alloy, such as NiTi, for either its super-elastic properties or its shape memory properties. When the coil 44 is made of shape memory alloy and is implemented for its shape memory properties, the portion of the coil made of shape memory alloy has a first configuration or state and a second configuration or state, which allows for the coil 44 to corkscrew into tissue. Accordingly, when the coil 44 is in one of the states and then heated, the coil returns to the other pre-defined state. In one arrangement, the coil has at least a portion that is a flat wire.

In a particular configuration, the coil 44 shown in FIG. 2 is made of NiTi (Nitinol). The Nitinol is super-elastic, shape memory alloy. When the coil 44 is above a transition temperature, which is approximately 10-15° C., it is in the austenitic state. In this state the coil 44 straightens out when retracted into the needle 28 due to a small amount of force incurred during retraction. When the coil 44 is advanced out of the needle 28, the coil 44 returns to its coil shape (or return to whatever shape it was shape set at).

FIGS. 3-1, 3-2, 3-3, 3-4 and 3-5 illustrate an example coil 44-1 that includes a straight section 50-1 and a coiled section 50-2 when in a deployed state. A cooling groove 52 is located on the side of the coil 44-1 that becomes the inside diameter surface of the coiled section 50-2 in the deployed state. The cooling groove 52 may be formed by an extrusion process, an etching process (e.g., laser), and/or a machining process. The width and depth of the cooling groove 52 may be a combination of various values, such that wicking/capillary action of the cooling fluid can occur.

As shown in FIGS. 4-1 and 4-2, a coil 44-2 includes three grooves 60 located on one side of the coil 44-2. The three grooves 60 may all have the same depth and width or the three grooves 60 may have different depths and widths. Also, the depths and widths of one or more of the three grooves 60 may vary along the length of the coil 44-2 or at different sections of the coil 44-2.

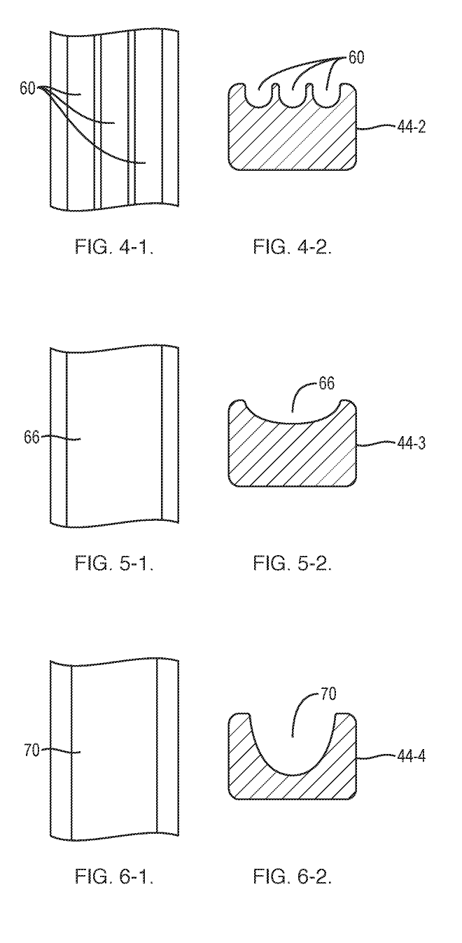

FIGS. 5-1 and 5-2 show a single grooved coil 44-3 with a groove 66 having width and depth. FIGS. 6-1 and 6-2 show a single grooved coil 44-4 with a groove 70 having a width value that is less than that of the coil 44-3 and a depth value that is greater than that of the coil 44-3.

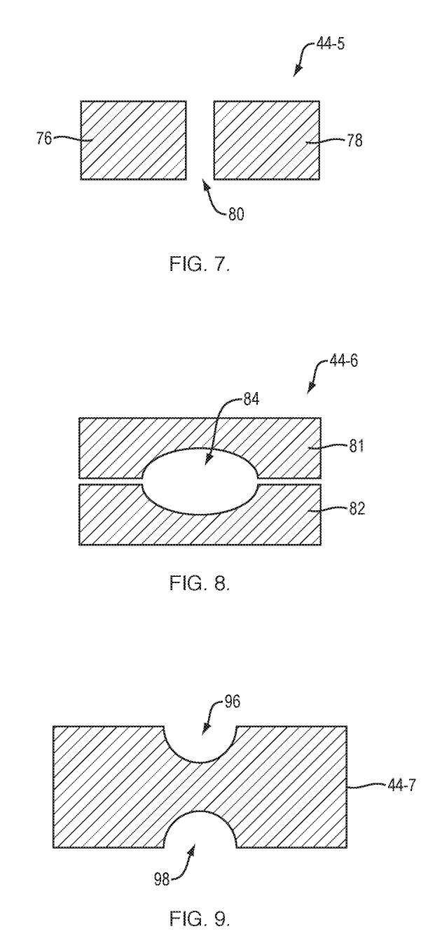

As shown in FIG. 7, a cross-sectional view of a coil 44-5 includes two adjacent sections 76, 78. Formed between the two sections 76, 78 is a cooling fluid transmission path 80. The two sections 76, 78 may be at the straight section and/or the curved section of coil 44-5 or may be present along only a portion of the straight section and/or the curved section. During cooling, fluid is received within the cooling fluid transmission path 80, whereby capillary action causes the fluid to maintain contact with at least a portion of the surfaces of the two sections 76, 78 on opposing sides of the cooling fluid transmission path 80. The opposing sides of the two sections 76, 78 may be convex, concave, grooved and/or etched.

FIG. 8 illustrates a cross-sectional view of a coil 44-6 similar to the coil 44-5. The coil 44-6 includes a first half 81 and a second half 82. Grooves are included in each of the halves 81, 82 along the sides of each half that oppose each other, thus forming a fluid flow path 84 at approximately the center of the coil 44-6. Fluid can either be pumped through the path 84, or it can wick through the channel 84 by capillary action. When fluid passes on the outside of the coil 44-6 proximal to the heated zone (coil) it will wick into the channel 84 by capillary action. When the fluid reaches the heated zone (coil) it will be forced out of the channel 84 by fluid expansion pressure.

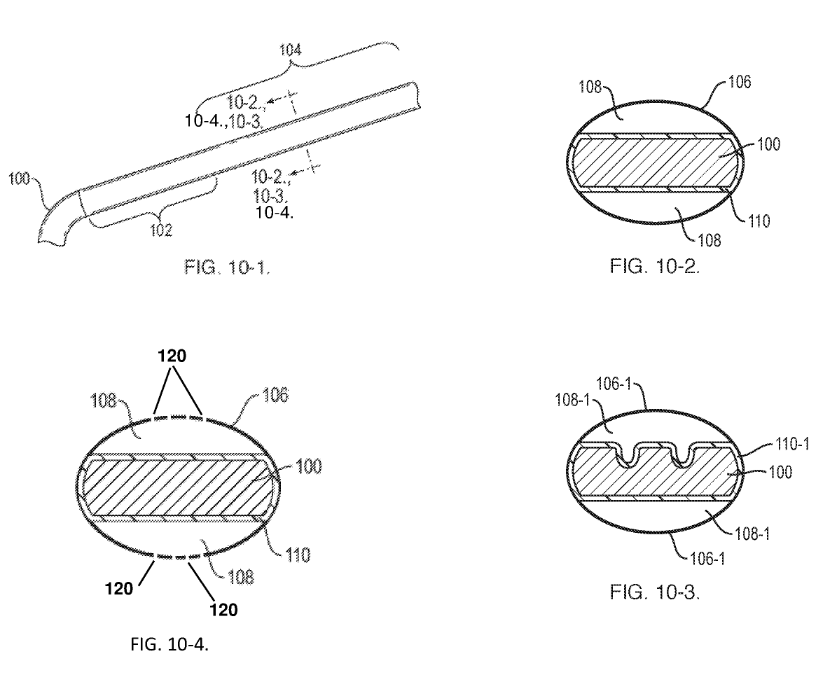

FIG. 9 illustrates a cross-sectional view of the straight section of a coil 44-7 and/or a coiled section of the coil 44-7. A first groove 96 is formed on a first side of the coil 44-7. A second groove 98 is formed on a second side of the coil 44-7. In the embodiment shown, the first and second grooves 96 and 98 are on opposing surfaces approximately at the center of the respective side. The grooves 96 and 98 may be offset from one another and may have different dimensions. In this configuration, wicking of fluid may occur on both sides of the coil 44-7 thus allowing for additional cooling. Other groove patterns may be used, such as crisscross, spiral, zigzag, etc.

FIG. 10-1 illustrates a partial view of a straight section of a coil 100. The straight section of the coil 100 includes a first section 102 that becomes exposed outside of a needle (not shown) after the coil 100 is deployed and the needle is retracted and a second section 104 that remains within the needle during operation. An insulation material (not shown) is applied from a distal end of the first section 102 all the way to the handle 26. A second material (not shown) is applied over the insulation material from at or near the distal end of the first section 102 to a proximal end of the second section 104. The second material produces one or more longitudinal gaps for allowing cooling fluid to flow from the proximal end of the section 102 to the distal end of the section 102. This is illustrated by the example cross-sectional views of FIGS. 10-2, 10-3 and 10-4.

As shown in FIG. 10-2, an insulation layer 110 is conformal to the outer surface of one embodiment of the coil 100. A second layer 106 is applied over the insulation layer 110 at sections 102 and 104 such that one or more gaps 108 are formed between the second layer 106 and the insulation layer 110. The insulation layer 110 may be formed of a thermoplastic polymer, such as PET—polyethylene terephthalate. The second layer 106 may include any type of heat shrink material.

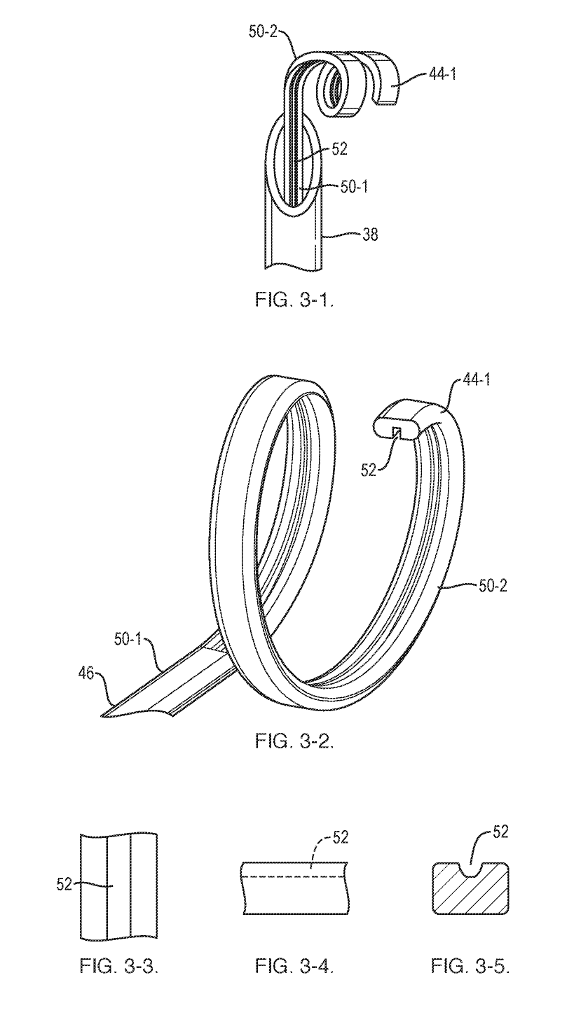

As shown in FIG. 10-3, an insulation material 110-1 is conformal to the outer surface of another embodiment of the coil 100 and may be similar to the insulation layer 110. The coil 100 includes two longitudinal grooves on one side. A second material 106-1 is applied over the insulation material 110-1 at sections 102, 104 such that one or more gaps 108-1 are formed between the second material 106-1 and the insulation material 110-1. The second material 106-1 may be any type of heat shrink material. The groove configurations shown in any of FIGS. 3 thru 9 may be used for the coil 100.

As shown in FIG. 10-4, the insulation layer 110 is conformal to the outer surface of one embodiment of the coil 100. The second layer 106 is applied over the insulation layer 110 at sections 102 and 104 such that one or more gaps 108 are formed between the second layer 106 and the insulation layer 110. The second layer 106 may include one or more micro holes 120 for allowing cooling fluid (e.g., saline) to perfuse into adjacent tissue. The micro holes 120 may be of any size (e.g., between 0.0005-0.002 inch (10-50 microns)).

The description of the invention is merely exemplary in nature and variations that do not depart from the gist of the invention are intended to be within the scope of the invention. Such variations are not to be regarded as a departure from the spirit and scope of the invention.

1. An electrosurgical instrument comprising:
a needle configured as a first electrode; and
a coil extending through the needle and configured as a second electrode, the coil being movable relative to the needle, the coil comprises:
a straight section;
a coil section; and
a fluid transmission feature,
wherein as the needle and the coil are inserted into tissue and energized with an electrical energy source, the needle and the coil apply current to the tissue to coagulate the tissue.

2. The electrosurgical device of claim 1, wherein the coil is electrically insulated from the needle.

3. The electrosurgical device of claim 1, wherein the fluid transmission feature comprises one or more grooves located on at least a portion of one or more sides of the coil section.

4. The electrosurgical device of claim 1, wherein the fluid transmission feature further comprises one or more grooves extending from a location on the straight section within the needle to a proximal end of the coil section.

5. The electrosurgical device of claim 1, further comprising a material applied over a portion of the straight section extending from a location on the straight section within the needle to a distal end of the straight section, the material forms one or more lumen having a first opening at a proximal end of the material and a second opening at a distal end of the material.

6. The electrosurgical device of claim 1, wherein at least one of the straight section or the coil section comprises two adjacent portions configured to form a fluid transmission path between the two portions.

7. The electrosurgical device of claim 6, wherein the fluid transmission feature comprises a first groove formed on a first side of one of the adjacent portions and a second groove formed on a second side of the other of the adjacent portions, wherein the first side and the second side are opposing.

8. The electrosurgical device of claim 1, wherein the coil is made of a super-elastic, shape memory alloy.

9. The electrosurgical device of claim 8, wherein the coil section returns to a coiled state when at least one of the coil is heated beyond a predefined transition temperature or the coil section exits a distal end of the needle.

10. A system for treating tissue comprising:
an energy source;
a fluid source; and
an electrosurgical instrument comprising:
a needle connected to the energy source, the needle being a first electrode, wherein the needle is in fluid communication with the fluid source; and
a coil extending through the needle and connected to the energy source, the coil being a second electrode and being movable relative to the needle, the coil comprising:
a straight section;
a coil section; and
a fluid transmission feature configured to wick fluid transmitted by the fluid source via the needle,
wherein as the needle and the coil are inserted into a target tissue and energized with the energy source, the needle and the coil apply current to the target tissue to coagulate the target tissue.

11. The system of claim 10, wherein the coil is electrically insulated from the needle.

12. The system of claim 10, wherein the fluid transmission feature comprises one or more grooves located on at least a portion of one or more sides of the coil section.

13. The system of claim 10, wherein the fluid transmission feature further comprises one or more grooves extending from a location on the straight section within the needle to a proximal end of the coil section.

14. The system of 13 claim 10, further comprising a material applied over a portion of the straight section extending from a location on the straight section within the needle to a distal end of the straight section, the material forms one or more lumen having a first opening at a proximal end of the material and a second opening at a distal end of the material.

15. The system of claim 10, wherein at least one of the straight section or the coil section comprises two adjacent portions, wherein the fluid transmission feature is defined by a space between the two portions.

16. The system of claim 15, wherein the fluid transmission feature comprises a first groove formed on a first side of one of the adjacent portions and a second groove formed on a second side of the other of the adjacent portions, wherein the first side and the second side are opposing.

17. The system of claim 10, wherein the coil is made of a super-elastic, shape memory alloy.

18. The system of claim 17, wherein the coil portion returns to a coiled state when at least one of the coil is heated beyond a predefined transition temperature or the coil portion exits a distal end of the needle.
